(19) United States
(12) Patent Application Publication
(10) Pub. No.: US 2011/0245737 A1
(43) Pub. Date: Oct. 6, 2011

(54) FETAL STIMULATION DEVICE

(75) Inventor: Dana Hawes, Richmond, VA (US)

(73) Assignee: PHRONESIS, LLC, Glen Allen, VA (US)

(21) Appl. No.: 13/075,231

(22) Filed: Mar. 30, 2011

Related U.S. Application Data
(60) Provisional application No. 61/282,786, filed on Mar. 31, 2010.

(51) Int. Cl. A61H 23/00; A41D 1/20; A41D 13/05; A41F 9/00; A41D 27/20; A61F 7/08; G09B 19/00

(52) U.S. Cl. 601/18; 2/69; 2/464; 2/311; 601/46; 434/236

(57) ABSTRACT

A fetal stimulation device is incorporated into a garment to be worn by a pregnant woman. The garment is preferably formed of an elastic material and includes a torso portion which is arranged around the torso of the woman. An audio device and a pair of speakers are connected with the garment torso portion to deliver sound such as music or words to a fetus. The garment torso portion also includes and elongated massaging element which is arranged around the perimeter of the torso portion to deliver vibrations to the fetus. The sounds and vibrations stimulate the fetus during prenatal development. In addition, massaging and heating elements are provided in the rear of the garment to deliver vibrations and heat to the back of the woman. A layer of radiation proof fabric is provided between the audio device and the woman to prevent radiation from reaching the fetus.

This application claims the benefit of U.S. provisional application No. 61/282,786 filed Mar. 31, 2010.

### BACKGROUND OF THE INVENTION

One of the most important choices adults will make is whether to bring a child into the world. When parents decide to step into parenthood, their lives will change. The life-changing process does not begin when the child is born, but rather as soon as he or she becomes a living, breathing entity in the womb. This prenatal period has proven to be one of the most influential to developing disposition, intelligence, and awareness. Many parents worry that they will not have enough time to devote to prenatal stimulation, especially if they are active members of the workforce, single parent, or even mothers who would like to maintain a level of fitness while pregnant. With the fetal stimulation device of the present invention, a mother is able to fulfill the emotional and physical needs of the child while also stimulating the child into early cognizance and higher intelligence.

### BRIEF DESCRIPTION OF THE PRIOR ART

There are a number of prenatal stimulation devices in the prior art. While they operate satisfactorily, they all suffer inherent drawbacks which limit their effectiveness. The present invention was developed in order to overcome these drawbacks by providing a stimulation device that combines the ability to deliver customized sounds, voices, and messages to a fetus while simultaneously providing comfort to the mother.

### SUMMARY OF THE INVENTION

Accordingly, it is a primary object of the invention to provide a fetal stimulation device including a garment worn by a pregnant woman and audio and massaging elements connected with the garment to deliver sound and vibrations to the fetus. More particularly, the garment includes a torso portion which extends over the torso when the garment is worn. The massaging element extends around the perimeter of the torso and a pair of speakers is provided in the bottom area of the torso portion.

According to a preferred embodiment, the garment is formed of a radiation shielding material to prevent radiation from the audio device from being directed toward the fetus. Alternatively, the audio device is arranged in a pocket formed in the garment. The pocket includes an inner layer of radiation shielding fabric to prevent radiation from impacting the fetus.

The back portion of the garment includes one or more heating elements and massage elements to deliver heat and vibration to the wearer. Further comfort is provided via an elastic support element of the garment which extends around the circumference of the garment and through an upper portion of the torso portion. A fetal monitoring unit is also connected with the garment, preferably opposite the audio device.

In an alternate embodiment, the fetal stimulation apparatus is incorporated into a belt worn about the torso of a pregnant woman. An audio device and massaging element are connected with the front portion of the belt to deliver sound and vibrations to the fetus, respectively. The fetus is shielded from radiation from the audio device by a layer of radiation blocking material. A plurality of spaced massaging elements and spaced heating elements are arranged in the back portion of the belt to deliver heat and vibrations to the woman.

### BRIEF DESCRIPTION OF THE FIGURES

Other objects and advantages of the invention will become apparent from a study of the following specification when viewed in the light of the accompanying drawing, in which

FIG. 1 is a front perspective view of a fetal stimulation device incorporated into a garment according to a preferred embodiment of the invention;

FIG. 2 is a side plan view of the device of FIG. 1;

FIG. 3 is a rear plan view of the device of FIG. 1;

FIG. 4 is a detailed sectional view of the pocket for holding an audio device according to a preferred embodiment of the invention;

FIG. 5 is a block diagram of a control circuit for the audio device and the heating and massage elements according to invention; and

FIG. 6 is a front plan view of a fetal stimulation device incorporated into belt according to an alternate embodiment of the invention.

### DETAILED DESCRIPTION

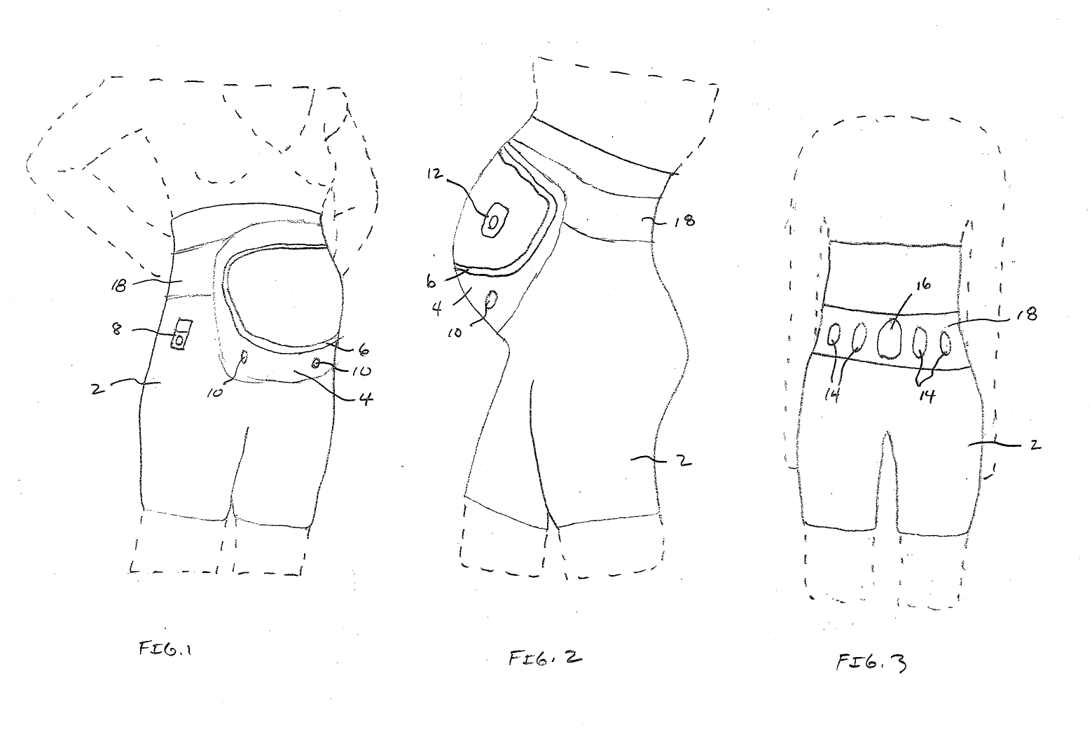

Referring to FIGS. 1-4, a first embodiment of the fetal stimulation device according to the invention will be described. In this preferred embodiment, the device includes a garment 2 which is worn by a pregnant woman. The garment is preferably formed of an elastic synthetic material which can be washed in a washing machine. The garment is in the form of a stretch leotard, but may have any configuration which can be comfortably worn by the user. Of most importance is that the garment includes a torso portion 4 which is arranged to surround the torso of the woman when worn as shown.

The torso portion includes an elongated massage device 6 which preferably extends around the perimeter of the torso portion as shown in FIG. 1. The massage device may be connected with the garment by any suitable means such as stitching. The device may include a plurality of spaced massaging transducers (not shown) arranged along its length. The device is operable to deliver vibrations to the torso area of the woman which mimic a massage. These vibrations provide comfort to the woman as well as a stimulus to the fetus arranged in the woman's womb.

In addition to providing vibration stimuli to the fetus, the invention also delivers sounds to the fetus. To this end, the garment includes an audio device 8 and at least one speaker 10. In the preferred embodiment shown in the drawing, a pair of speakers is arranged toward the bottom of the torso portion of the garment. The speakers are spaced on either side of the woman's torso to deliver music or voice to the fetus.

The audio device 8 uses the latest compressed audio technology to allow the parent to download customizable musical playlists from a secure product web site as well as to record and play stories and soothing words, all in the parent's own voice. This introduces the child to his parent before the child is born.

As shown in FIG. 2, a fetal monitor 12 is connected with the torso portion 4 of the garment. The monitor allows the mother to monitor the condition of the fetus. Using wireless communication technology, signals from the fetal monitor may also be delivered to a remote computer or to a doctor's office where the signals are stored for later analysis.

Referring to FIG. 3, additional massage pads 14 are connected with the rear of the garment. In addition, one or more heating pads 16 are also connected with the rear of the garment. The massage and heating pads deliver heat and vibration to the lower back area of the woman to provide additional comfort.

A unique feature of the garment is that it may be formed of a radiation shielding fabric. This enables the mother to safely use the stimulation device according to the invention while shielding the fetus from radiation which could be emitted from the various components connected with the garment, particularly the audio device.

The garment also includes an elastic support 18 which extends around the circumference of the garment and through an upper portion of the torso portion. The massage pads 14 and heating elements 16 are arranged within the support. In this regard, the elastic support is preferably provided with pockets for receiving the heating and massage pads.

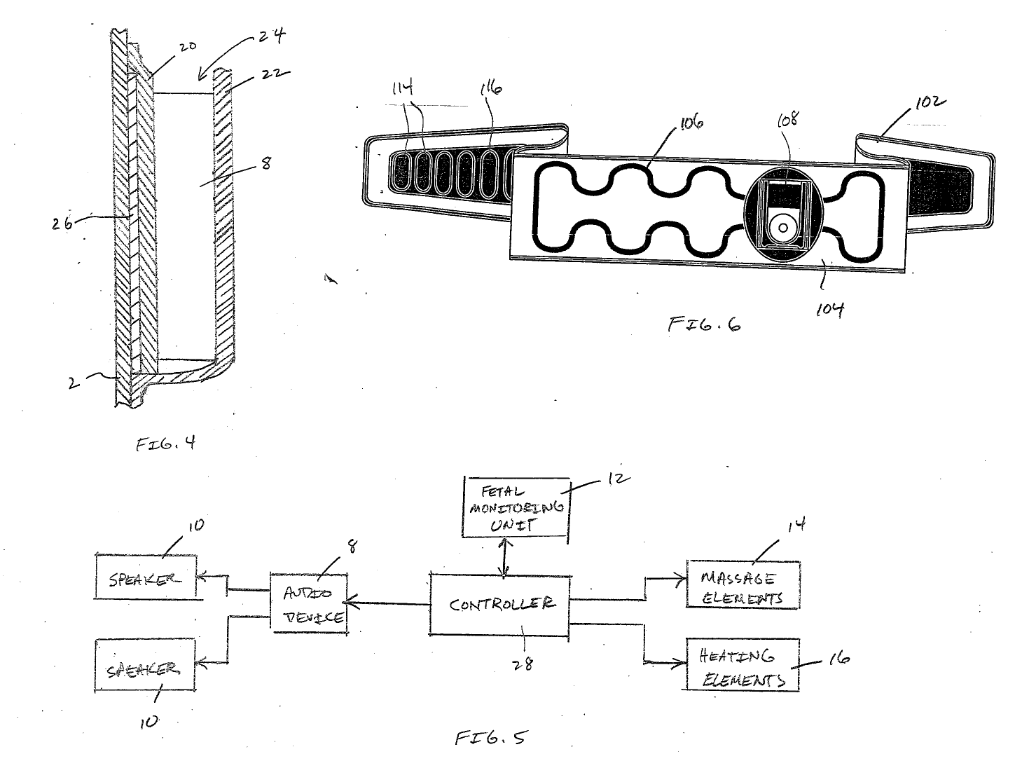

The audio device is preferably arranged in a pocket provided in the garment as shown in FIG. 4. The pocket includes inner 20 and outer 22 layers of fabric which are stitched to the garment 4. An opening is provided between the upper edges of the inner and outer layers to define the pocket 24 for receiving the audio device 8. To further protect the fetus from radiation, a layer of radiation shielding material 26 is provided between the inner pocket fabric layer 18 and the garment 4.

FIG. 5 is a block circuit diagram illustrating the operation of the stimulation elements of the invention. A controller 28 is provided to control the operation of the various elements. The controller is connected with the audio device 8 which in turn is connected with the speakers 10 to deliver sound or voice to the fetus. In a practical embodiment, the controller may be incorporated directly into the audio device to control the selection, volume, and duration of sound delivery. The massage 14 and heating 16 elements are also connected with the controller to control the intensity and duration of each. The fetal monitoring unit is also connected with the controller to deliver signals from the fetus. As noted above, the connection between the monitoring unit and the controller may be a wireless connection.

An alternate embodiment of the invention is illustrated in FIG. 6. As shown therein, the invention includes a belt 102 that is adapted to be worn by a pregnant woman so that the belt is arranged over the torso of the woman to surround the womb. The belt is fastened by conventional means such as hook and pile fasteners, buckles, straps, or the like. The belt includes a torso portion 104 which includes an elongated massaging element 106 and an audio playing device 108. These are arranged adjacent to the torso and womb when the belt is worn. At the rear of the belt are provided massaging elements 114 and at least one heating element 116 to deliver vibrations and heat to the lower back region of the woman.

With the stimulation device according to the invention, the fetus is provided with sound and vibration stimuli under the control of the mother to stimulate development and growth. The mother is simultaneously provided with an intensive massage and heat in the lower back area to eliminate stress and pair directly where pregnant women most need relief. Adjustable straps (not shown) insure comfort to the wearer regardless of the size or position of the fetus. The audio elements and massage pads are preferably removable from the device so that the garment may be washed as necessary.

By allowing the mother to move freely without losing sleep because of her unborn child, the subject invention provides relief during pregnancy. The fetal stimulation device allows the mother to take a walk, relax on a couch, and cook dinner while receiving an invigorating, deep muscle massage, all while allowing her fetus to develop into a more intelligent, cognizant, and stable child.

While the preferred forms and embodiments of the invention have been illustrated and described, it will become apparent to those of ordinary skill in the art that various changes and modifications may be made without deviating from the inventive concepts set forth above.

What is claimed is:

1. A fetal stimulation device, comprising
(a) a garment adapted to be worn by a pregnant woman, said garment including a torso portion extending over the torso of the woman;
(b) an audio device connected with said garment torso portion for delivering sound to a fetus within the torso; and
(c) at least one massaging element connected with said garment torso portion for delivering vibrations to the torso.

2. A fetal stimulation device as defined in claim 1, wherein said massaging element extends around the perimeter of the torso portion.

3. A fetal stimulation device as defined in claim 1, wherein said garment is formed of a synthetic elastic fabric.

4. A fetal stimulation device as defined in claim 3, wherein said fabric is formed of a material which prevents the transmission of radiation.

5. A fetal stimulation device as defined in claim 3, wherein said garment includes a pocket for receiving said audio device.

6. A fetal stimulation device as defined in claim 5, wherein said pocket includes a layer of material between said audio device and the woman which prevents the transmission of radiation.

7. A fetal stimulation device as defined in claim 1, and further comprising at least one speaker connected with said audio device, said speaker being arranged in a bottom area of said torso portion.

8. A fetal stimulation device as defined in claim 7, and further comprising a spaced pair of speakers.

9. A fetal stimulation device as defined in claim 2, and further comprising a plurality of spaced massaging elements arranged in a back portion of said garment.

10. A fetal stimulation device as defined in claim 9, and further comprising a plurality of spaced heating elements arranged in a back portion of said garment.

11. A fetal stimulation device as defined in claim 10, wherein said garment includes an elastic support element extending around the circumference of said garment and through an upper portion of said torso portion, said plurality of spaced massaging elements and heating elements being arranged within said elastic support element.

12. A fetal stimulation device as defined in claim 1, and further comprising a fetal monitoring unit connected with said garment torso portion.

13. A fetal stimulation device, comprising
(a) a belt adapted to be worn by a pregnant woman and extending over the torso of the woman;
(b) an audio device connected with a front portion of said belt for delivering sound to a fetus within the torso; and
(c) at least one massaging element connected with a said belt front portion for delivering vibrations to the torso.

14. A fetal stimulation device as defined in claim 13, wherein said belt includes a layer of material between said audio device and the woman to prevent radiation from said audio device from being transmitted to the fetus.

15. A fetal stimulation device as defined in claim 14, and further comprising a plurality of spaced massaging elements arranged in a back portion of said garment.

16. A fetal stimulation device as defined in claim 15, and further comprising a plurality of spaced heating elements arranged in a back portion of said garment.
